(19) United States
(12) Patent Application Publication
(10) Pub. No.: US 2018/0235642 A1
(43) Pub. Date: Aug. 23, 2018

(54) SURGICAL TOOL GUIDE

(71) Applicant: Imperial Innovations Limited, London (GB)

(72) Inventors: Andrew Amis, London (GB); Jianmo Li, London (GB)

(21) Appl. No.: 15/752,805

(22) PCT Filed: Aug. 12, 2016
(86) PCT No.: PCT/GB2016/052514

§ 371 Date: Feb. 14, 2018

(30) Foreign Application Priority Data
Aug. 14, 2015 (GB) 1514534.5

(51) Int. Cl. A61B 17/17; A61B 17/16; A61B 34/10; A61B 17/56; A61B 17/15

(52) U.S. Cl. CPC A61B 17/1764; A61B 17/1675; A61B 17/154; A61B 2017/568; A61B 2034/108; A61B 34/10

(57) ABSTRACT

A surgical tool guide (100) for minimally invasive preparation of a bony surface to receive an implant comprises at least one contact surface (104, 120) for securely registering the surgical tool guide (100) on a bone (200), and a mechanical guide means (102) configured to restrict movement of a surgical tool (110) received therein such that a working tip (111) of the surgical tool (110), in use, is guided over a predefined area of the bone (200), typically at a pre-planned depth.

### FIELD OF THE INVENTION

The present invention relates generally, but not exclusively, to surgical tool guides, in particular, to minimally-invasive patient-specific surgical tool guides for use in orthopaedic surgery, being securely registrable to a patient for guiding a surgical tool over a pre-operatively defined path.

### BACKGROUND TO THE INVENTION

Existing patient-specific surgical tool guide designs comprise an integrated large-piece registration guide and surgical tool guide (see, for example, US 2011/0106093 A1). The rationale behind such surgical guide designs is that the larger the guide, the larger the number of surface features that can be located, meaning that the guide can be more accurately registered, and with greater stability. The result is that the guide is much less likely to be secured in an inaccurate position or otherwise become dislodged during surgery, meaning that surgical procedures can be carried out with greater accuracy.

Although increased stability and accuracy of placement can be achieved using larger surgical guides with larger contact surfaces, there are problems associated with the use of such guides.

Such larger guides can only be used in open surgery. For example, in orthopaedic surgery, where the surgical guide needs to be registered on to the bone of a patient, a substantial amount of the patient's bone needs to be exposed so that the large guide can be securely registered thereon. This is can result in damage to healthy tissue, increased hospitalisation time and increased patient discomfort and recovery time. There is also an increased risk of infection due to the structures and tissues being directly exposed to air during an open surgical procedure.

Accordingly, there is a need for a surgical tool guide which can provide the increased stability of a larger surgical tool guide and whilst avoiding the above outlined problems associated with the use of such guides.

Furthermore, existing patient-specific surgical tool guide designs, such as the guide described in US 2011/0106093 A1, define a guide path adjacent the bone surface, whereas a surgeon using the guide manipulates the surgical tool outside the body, remote from the bone surface. This means that a working tip of the tool is prone to ‘wobble’, bend, or be deflected from its intended path; for example, where the surgeon varies the angle of the surgical tool relative to the surgical tool guide, consequently varying the angle of the working tip. This can lead to inaccuracies in the surgical procedure which can have a significant impact on the success of the procedure.

Accordingly, there is a need for a surgical tool guide which eliminates the risk of ‘wobble’ during a surgical procedure.

In addition, if the intention is for the surgery to be performed minimally-invasively, it is then not possible to have a guide resting directly onto the wide extent of the bone or joint surface to be prepared as such a guide would require a significant amount of the bone or joint surface to be exposed during surgery.

Accordingly, there is a need for a surgical tool guide which is suitable for use in minimally-invasive surgery.

### SUMMARY OF INVENTION

According to a first embodiment, there is provided a surgical tool guide for minimally invasive preparation of a bony surface to receive an implant comprising: at least one contact surface for securely registering the surgical tool guide on a bone; and a mechanical guide means configured to restrict movement of a surgical tool received therein such that a working tip of the surgical tool, in use, is guided over a predefined area of the bone.

The mechanical guide means is extracorporeal meaning that, in use, it is located outside of the body of the patient on which the surgical tool guide is being used.

In the embodiment where there is only a single contact surface, the contact surface may be designed such that it enables secure registering of the surgical tool guide on a bone. For example, the contact may be shaped to ‘clip’ onto the bone surface or fixing means may be used to ensure a stable engagement with the bone.

Advantageously obviates the need to expose large parts of the bone surface when registering the surgical tool guide to the bone.

Further, operations carried out using the surgical tool guide are greatly simplified as movement of the surgical tool is restricted in such a way that the working tip of the surgical tool cannot be moved outside of the area of the bone which is to be operated on by the surgical tool, greatly reducing the margin for error.

Preferably, the mechanical guide means is further configured to restrict movement of the surgical tool to a predetermined depth when in use.

Advantageously, operations carried out using the surgical tool guide are greatly simplified as movement of the surgical tool is restricted in such a way that the working tip of the surgical tool cannot be moved beyond the predetermined depth, greatly reducing the margin for error.

Preferably, the contact surface is configured to register the surgical tool guide in a unique position on the bone surface.

Advantageously, this ensures that the surgical tool guide can easily be registered in the correct position, greatly reducing the margin for error.

Preferably, there are a plurality of contact surfaces.

Advantageously, this increases the stability of the surgical tool guide.

Preferably, there are three contact surfaces.

Preferably, the mechanical guide means is configured, in use, to constrain movement of the working tip of the surgical tool to a three dimensional volume of space defined in a pre-surgical planning phase.

Advantageously, operations carried out using the surgical tool guide are greatly simplified as the working tip of the surgical tool is constrained to moving within a three dimensional volume of space defined in a pre-surgical planning phase, greatly reducing the margin for error.

Preferably, the mechanical guide means comprises a surface contoured to guide the working tip to produce a predefined bone surface geometry.

Preferably, at least one of the contact surfaces is removably attachable to the mechanical guide means.

Preferably, each of the contact surfaces is removably attachable to the mechanical guide means in such a way that the surgical tool guide can only be assembled in a single configuration.

Preferably, each of the contact surfaces comprises a contoured pad disposed at a distal end of a contact arm relative to the mechanical guide means.

Preferably, one or more of the contoured pads is configured to contact a unique position on a bone surface.

Preferably, one or more of the contact surfaces comprises a fixing means selected from a group comprising: wires, pins and screws, securable to the bone,

Preferably, said fixing means includes depth stops or spacers to locate the mechanical guide means at a known height above the bone.

Preferably, the mechanical guide means comprises a proximal portion and a distal portion, wherein the proximal portion comprises a guide surface to restrict the depth and position of the surgical tool when in use.

Preferably, the distal portion comprises a pivot means, such as a bearing, to restrict movement of the surgical tool when in use.

Preferably, the guide surface comprises one or more slots through which a surgical tool can be inserted.

Preferably, the guide surface comprises a groove into which a part of the cutting device can be inserted when in use.

Preferably, the distal portion comprises an aperture through which a surgical tool is inserted when in use.

Preferably, the distal portion comprises a bearing containing said aperture.

Preferably, each of the contact surfaces are designed to pass through respective incisions.

Preferably, the surgical tool guide is manufactured on the basis of patient-specific data.

Preferably, the surgical tool guide is manufactured using an additive manufacturing process.

Preferably, the mechanical guide means comprises a removable guide plate which can be interchanged with at least one other guide plate.

According to a second embodiment, there is provided a surgical kit comprising: the surgical tool guide of any preceding claim; and a surgical tool configured to engage with the mechanical guide means of the surgical tool guide.

Preferably, the surgical tool comprises a protrusion for abutment against the groove of the mechanical guide means.

Preferably, the mechanical guide means comprises a collar received in the slot, and wherein the collar is configured to limit the depth of the surgical tool therein.

According to a third embodiment, there is provided a method of registering a surgical tool guide on a bone, the method comprising the step of: making at least one arthroscopic incision in tissue adjacent to the bone; and inserting a respective contact surface of the surgical tool guide through said at least one arthroscopic incision such that the one or more contact surfaces contact a bone surface in such a way as to securely register the surgical tool guide on the bone.

According to a fourth embodiment, there is provided a method of preparing a bone surface to receive an implant, the method comprising the steps of: securely registering a surgical tool guide on a bone; engaging a surgical tool with a mechanical guide means of the surgical tool guide; and preparing the bony surface to receive an implant using the surgical tool, wherein movement of a working tip of the surgical tool is constrained, by the mechanical guide means, to a volume of space defined in a pre-surgical planning phase.

According to a fifth embodiment, there is provided a method of manufacturing the surgical tool guide of the first embodiment, comprising the steps of: designing the at least one contact surface on the basis of patient-specific data such that the surgical tool guide is securely registrable in a predefined position and orientation on the patient's bone; designing the mechanical guide means on the basis of patient-specific data such that a working tip of a surgical tool, in use, is guided over a predefined area of the patient's bone defined in a pre-surgical planning phase; and manufacturing the surgical tool guide according to said designing steps.

### BRIEF DESCRIPTION OF THE DRAWINGS

Embodiments of the present invention will now be described, by way of example only, with reference to the accompanying drawings, in which:

FIG. 1a depicts a surgical tool guide;

FIG. 1b depicts an alternative surgical tool guide;

FIG. 2 depicts an exemplary configuration of a slot and groove in cross-section;

FIG. 3a depicts an exemplary guide surface;

FIG. 3b depicts a further exemplary guide surface;

FIG. 3c depicts a further exemplary guide surface; and

FIG. 3d depicts a further exemplary guide surface.

### DETAILED DESCRIPTION

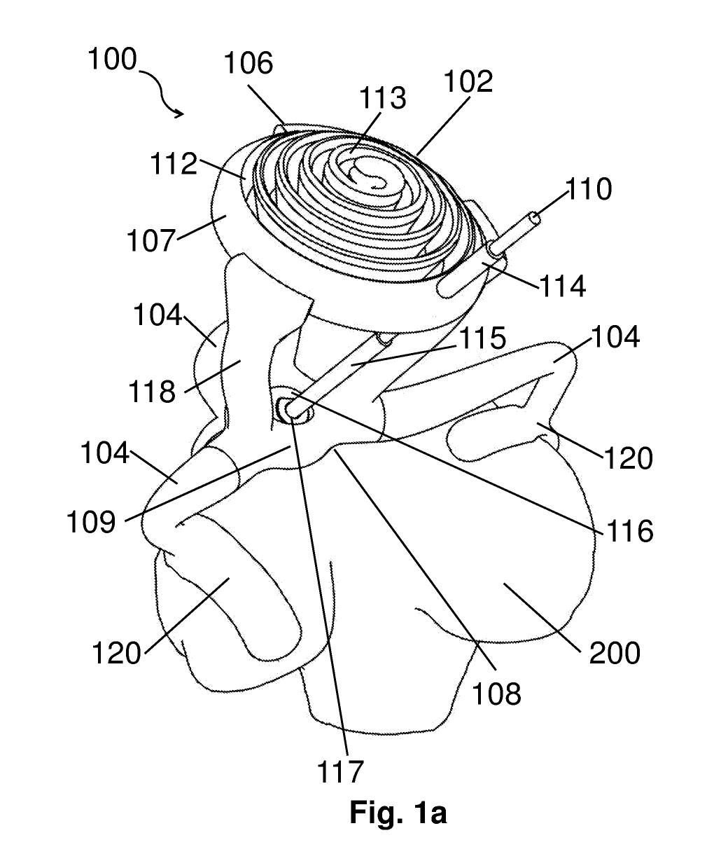

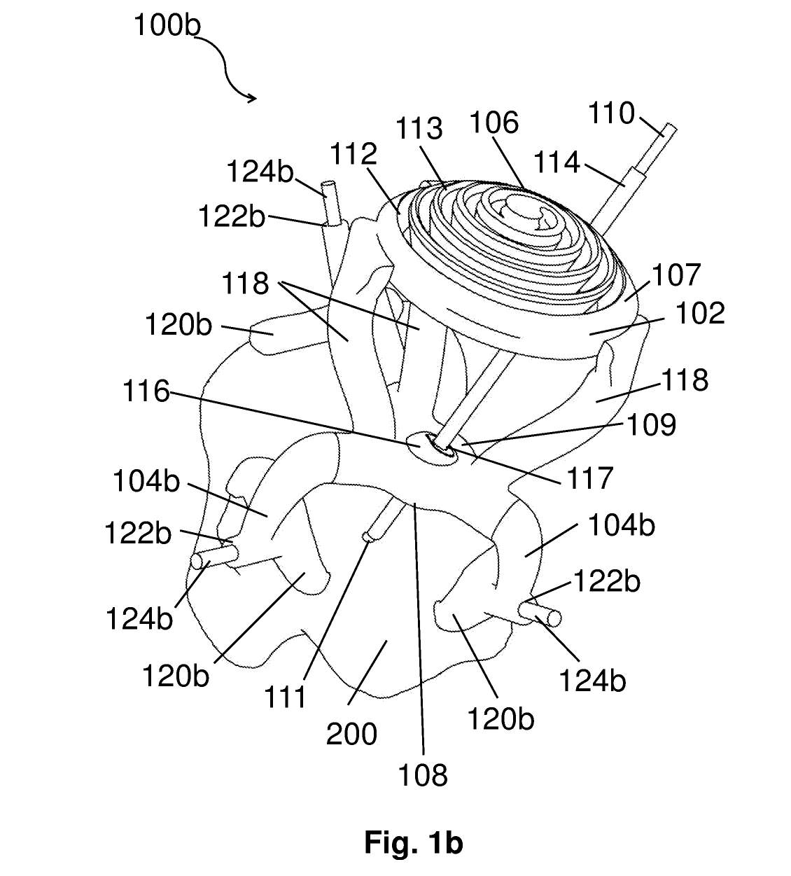

FIG. 1a depicts a surgical tool guide 100 according to one embodiment. The surgical tool guide 100 shown in FIG. 1a comprises a guide means 102 securely attached to a registration portion comprising three contact arms 104, each with a contoured pad 120 disposed at a distal end relative to the mechanical guide means 102.

Together, each of the contact arms 104 and its corresponding contoured pad 120 are known as contact surfaces.

The surgical tool guide 100 shown in FIG. 1a is registered to the distal end of a femur 200. It will be understood that, although the surgical tool guide 100 is shown registered to the distal end of a femur 200, the surgical tool guide 100 can be configured to register to any part of any bone, as will be made clear in what follows.

The contact arms 104 and corresponding contoured pads 120 are configured such that the surgical tool guide 100 can be securely registered to the bone 200 in a unique pre-defined position, by virtue of bottom surfaces of each of the contoured pads 120 being shaped and configured to match the contours of respective corresponding portions of the surface of the bone 200.

The contoured pads 120 may be designed based on the basis of patient-specific data obtained during a pre-surgical planning phase, for example, by scanning a patient's bone using suitable scanning equipment and creating a computer model of the bone 200 based on the results of the scan.

The contours of the surfaces of bone 200, or of the surface of an articular joint composed of articular cartilage which overlays the bone, may be determined pre-operatively, through techniques including the use of computer-assisted image methods based on three-dimensional images of the patient's anatomy reconstructed from MRI, CT, ultrasound, X-ray, or other three- or two-dimensional medical scans of the patient's anatomy.

Once the contours of the surfaces of bone 200 have been accurately determined, a model of the bone 200 can be created on a computer using any suitable known modelling software. The optimum placement of the surgical tool guide 100 can then be determined and the contact arms 104 and the contoured pads 120 can be designed accordingly, for example, by also creating a model of the surgical tool guide 100 on a computer. Using the computer model of the bone 200, the contoured pads 120 can be designed to fit onto a particular position on the surface of bone 200.

The surgical tool guide 100 may then be manufactured based on the computer model of the surgical tool guide 100 using, for example, a suitable form of rapid prototyping. For example, any suitable additive or subtractive manufacturing process may be used to manufacture the surgical tool guide 100.

As shown in FIG. 1a, the surface of each of the contoured pads 120 which contacts the surface of the bone 200 is contoured to exactly match a specific associated area of the surface of the bone 200. The contoured pads 120 are arched with a generally concave contoured surface. The contoured pads ‘hook’ onto/around corresponding convex contours on the surface of the bone 200. The contoured pads 120 shown in FIG. 1a are respectively designed to match a unique portion of the medial and lateral condyles of the femur 200.

The contact arms 104 are substantially L-shaped and meet centrally at a lower portion of the guide means 102, as shown in FIG. 1a.

The contact arms 104 are configured such that each of the contoured pads 120 is held in a position relative to the other contoured pads 120 in such a way that the contoured pads 120 simultaneously line up with and can be placed on the specific area of the surface of the bone 200 which they are contoured to exactly match (i.e. the medial condyle, lateral condyle and trochlear groove).

Each of the contact arms 104 may be configured to pass through a respective arthroscopic incision. Small incisions may be made at the locations where each of the contoured pads is to be registered to the surface of the bone 200. The contoured pads 120 may each be inserted into their respective incision and registered to the surface of the bone 200.

The following example steps may be followed when registering the surgical tool guide 100 to the surface of a bone 200:

1. A pre-operative plan is made using computer models of the bone 200 and surgical tool guide 100;

2. Small incisions are made on the patient in accordance with the pre-operative plan;

3. The contoured pads 120, connected to the contact arms 104 are inserted through the incisions (the contoured pads 120 may be inserted through the incisions first and subsequently connected contact arms 104, where they are detachable from the contact arms 104);

4. Once in place, the contact arms 104 are assembled to guide means 102, where the contact arms are detachable from the guide means 102;

5. The surgeon ensures that the surgical tool guide 100 is in the registration position determined during the pre-operative planning phase;

6. The operation is then carried out.

Advantageously, the surgical tool guide 100 obviates the need to expose large parts of the bone surface when registering the surgical tool guide 100 to a bone.

Also or instead, the contoured pads 120 may be detachable from the contact arms 104 and may be inserted into the respective arthroscopic incisions prior to each contact arm 104 being attached to its respective contoured pad 120.

It will be understood that alternative arrangements of the surgical tool guide 100 are envisaged. For example, although the contact arms 104 and the contoured pads 120 shown in FIG. 1a are configured such that the surgical tool guide 100 can be uniquely registered on the surface in the position shown in FIG. 1a, the contact arms 104 and the contoured pads 120 may be configured such that the surgical tool guide 100 can be uniquely registered in a different position on the surface of a different part of the same bone 200.

Each of the contact arms 104 may also or instead comprise a fixing means for fixing each contoured pads 120 to the surface of the bone 200. Suitable fixing means include, but are not limited to being one of: wires, pins and screws capable of securing the contoured pads 120 to the bone 200. The design of the contoured pads 120 may be altered such that they are suitable for use with the particular fixing means used.

The fixing means may include gap stops or spacers to locate the mechanical guide means 102 at a known height above the bone 200.

FIG. 1b depicts a surgical tool guide 100b which differs from the surgical tool guide depicted in FIG. 1a in that the contact arms 104b and contoured pads 120b of the surgical tool guide 100b further comprise an aperture 122b through which a fixing means 124b is inserted. The fixing means shown in FIG. 1b comprises a pin 124b which secures the associated contoured pad 120b to the surface of the bone 200 in a fixed position.

The following example steps may be followed when fixing the surgical tool guide 100b to the surface of a bone 200:

1. A pre-operative plan is made using computer models of the bone 200 and surgical tool guide 100;

2. Small incisions are made on the patient in accordance with the pre-operative plan;

3. The contoured pads 120, connected to the contact arms 104 are inserted through the incisions (the contoured pads 120 may be inserted through the incisions first and subsequently connected contact arms 104, where they are detachable from the contact arms 104);

4. Once in place, the contact arms 104 are assembled to guide means 102, where the contact arms are detachable from the guide means 102;

5. The surgeon ensures that the surgical tool guide 100 is in the registration position determined during the pre-operative planning phase;

6. The fixing means 124b in the apertures 122b are drilled into the bone 200, fixing the surgical tool guide 100 to the bone 200.

7. The operation is then carried out.

The fixing process is simple. After the surgical tool guide 100 is placed in the correct registration position, the fixing means 124b are drilled, by rotational machine, into the bone 200 through the apertures 122b in the contoured pads 120b.

No pilot holes are needed as the direction of each of the fixing pins is guided by the holes in the aperture 122b in the contoured pads 120b.

There are various type of fixing pins which can be used, including threaded and smooth ones. The surgeon will choose the most suitable one according to the patient's bone condition. But commonly, the smooth fixing pins are sufficient to fix the surgical tool guide 100 to the bone surface.

One or more of the contact arms 104 may be removably attachable to the guide means 102, for example, via a removable press fit engagement or via a snap fit engagement. The attachment may be configured such that the contact arms 104 can only be attached to the guide means 102 in such a way that the surgical tool guide 100 can only be assembled in a single configuration.

The contoured pads 120 may be removably attachable to the contact arms 104. The attachment between the contoured pads 120 and the contact arms 104 may be configured such that the contoured pads 120 can only be attached to their respective contact arms 104. The attachment may be configured such that the contoured pads 120 and contact arms 104 can only be assembled in a single configuration.

Alternatively, each contoured pad 120 and its associated contact arm 104 may be made as a single, unitary piece, with the end of the contact arm 104 opposite the contoured pad 120 configured to allow assembly to the guide means 102 in a unique position.

Although the embodiments of the surgical tool guide 100 shown in FIGS. 1a and 1b each comprise three contact arms 104, it will be understood that any suitable number of contact arms 104 may be used.

For example, a single contact arm 104 may be used provided the associated contoured pad 120 has sufficient surface contact area and topology to securely register the surgical tool guide 100 to the bone 200 in a unique position.

Alternatively, the surgical tool guide 100 may comprise one or more contact arms 104, which each split into two or more branches, with a contoured pad 120 attached to the distal end of each branch.

The mechanical guide means 102, comprises a proximal portion 106 and a distal portion 108. The proximal portion 106 comprises a dome-shaped member 107 spaced from the distal portion 108 by three connecting arms 118. A spiral shaped slot 112 is formed in the dome-shaped member 107. The slot is grooved, with grooves 113 on either side thereof. The grooved slot 112 constitutes a guide surface, which is configured to guide a working tip 111 of a surgical tool 110 during a surgical procedure, by interaction with a corresponding guiding feature (such as a collar, or abutment portion 114) on the surgical tool 110.

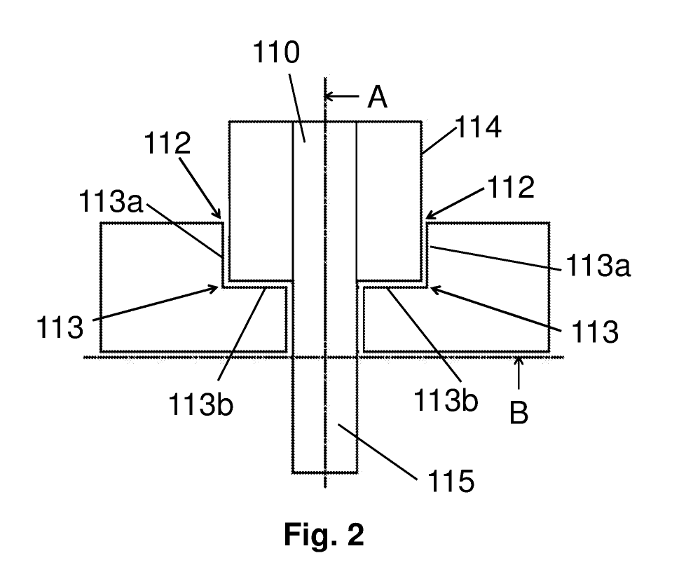

In the illustrated exemplary embodiments, the guide surface comprises the grooved slot 112 and the guiding feature on the surgical tool 110 comprises an abutment portion 114. Their interaction will be described in more detail in connection with FIG. 2.

The surgical tool 110 comprises a shaft 115 with a working tip 111 at its distal end and cylindrical abutment portion, or collar, 114 towards its proximal end.

The collar 114 sits within the grooves 113 in the slot 112 and is wider in diameter than the width of the slot 112 at its narrowest point between directly opposing grooves 113. As such, movement of the surgical tool 110 through the slot 112 is restricted by the collar 114 because the collar 114 is unable to pass beyond the grooves 113 of the slot 112.

FIG. 2 depicts an exemplary configuration of the slot 112 and groove 113 in cross-section. Each groove 113 comprises a wall portion 113a extending parallel to the common longitudinal axis A of the surgical tool 110. The grooves 113 also comprise a base portion 113b disposed at an end of the wall portion 113a closest to the distal portion 108 of the guide means 102. The base portions 113b of the grooves 113 extend along axis B, perpendicular to axis A (the longitudinal axis of the surgical tool 110) and parallel to a virtual line across the narrowest point between the two sides of the slot 112.

For each section of the slot 112, the direction of the axis A of the cutter is calculated to ensure that the base portions 113b, on both sides of the slot 112, are perpendicular to the axis A of the surgical tool 110. This provides more accurate control of surgical tool 110 depth, leading to better accuracy in the resultant shape of the bone surface.

The shortest distance between base portions 113b of the slot 112 is narrower than the diameter of the abutment 114, but wider than the diameter of the shaft 115 of the surgical tool 110 so as to allow passage of the distal end of the shaft 115 through the slot 112, as shown in FIG. 2.

The width of the slot 112 defined by the wall portions 113a of the grooves 113 is fractionally larger than the diameter of the collar 114 so as to allow translational movement of the collar 114 along the slot 112 whilst restricting any transverse movement of the collar 114 along axis B. Moreover, off-axis ‘wobble’ of the shaft 115 may be prevented by the engagement of the collar 114 against the wall portions 113a of the grooves 113; wherein taller wall portions (and a correspondingly tall collar) minimise any such off-axis movement.

The distal portion 108 of the mechanical guide means 102, as shown in FIGS. 1a and 1b, comprises a bearing 116 housed within a bearing housing portion 109. The outer surface of bearing 116 is spherical and is free to rotate within the bearing housing portion 109. The bearing 116 comprises an aperture 117, which may be cylindrical, through its centre and through which the shaft 115 of the surgical tool 110 can be inserted. The aperture 117 may intersect the centre of the bearing 116 and/or the point about which the bearing 116 rotates within the bearing housing portion 109.

The aperture 117 is of uniform diameter along its length. The width of the aperture 117 is fractionally larger than the diameter of the shaft 115 of the surgical tool 110 which passes through the aperture 117, such that axial movement of the surgical tool through the aperture is permitted, but lateral movement of the surgical tool 110 relative to the bearing 116 is constrained.

Movement of the surgical tool 110 is constrained, at two locations, by the combination of the grooved slot 112 in conjunction with the abutment portion or collar 114 of the surgical tool 110, and the aperture 117 in the bearing 116 in conjunction with the portion of the shaft 115 of the surgical tool 110 that passes through it.

As shown in FIGS. 1a and 1b, the proximal portion 106 of the surgical tool guide 100 is connected to the distal portion 108 of the mechanical guide means 102 via three connecting arms 118. The length of the connecting arms 118, together with the configuration of the guide surface, the position of the abutment portion 114 along the shaft 115 of the surgical tool 110, the bearing 116 and the configuration of the contact arms 104, constrains the maximum depth of the working tip 111 of the surgical tool 110 throughout the permitted range of movement of the surgical tool 110.

More generally, the guide is designed such that when a guiding feature on a surgical tool 110 is passed over the guide surface, a defined, desired pathway of the surgical tool 110 working tip 111 is followed. Preferably, that defined pathway is further constrained by means of the tool 110 also passing through an aperture (such as the aperture 117 through the bearing 116), at a more distal position than the guide surface.

Accordingly, the area over which the tip 111 of the surgical tool 110 must pass is constrained. As such, the tip of the surgical tool 110 necessarily works across an entire pre-defined surface of the bone.

This mechanism ensures that no part of the defined bone surface is missed, and that every part of that defined surface is machined to a pre-defined depth. Thus, by simply following the guidance built into the surgical tool guide 100, a surgeon may easily create an accurate pre-defined three-dimensional surface in the bone.

By designing a suitable three-dimensional guide surface, the depth and position of the working tip 111 may be restricted and controlled to both cover a desired surface area on the bone and also to have controlled, possibly variable depth. By this means, complex three-dimensional geometries may be created in the bone. By way of example, if a section of bone is to be replaced by a layer of repair material that matches the original articular geometry, then if that repair material is to have an even thickness, the surface traversed and machined by the tool tip 111 would be defined as a surface matching but offset from the original articular surface by that depth.

A general principle of the surgical tool guide 100 is that the guide means 102 is positioned outside of the body (i.e. the guide means 102 is extracorporeal), while the surgical tool 110 position is controlled via a pivot means (e.g. bearing 116) close to the bone surface. This controls the orientation of the surgical tool 110, eliminating ‘wobble’ inaccuracy. It also means that the transverse movement of the tool tip 111 is scaled-down by the ratio of the lengths between the guide surface and the pivot means, versus the lengths between the pivot means to the tool tip 111 at the bone surface.

Because the guiding pivot means 116 is close to the skin surface when the surgical tool guide 100 is used in minimally invasive surgery, transverse movement of the shaft 115 of the surgical tool 110 at the skin surface will be very small. This allows the surgical tool 110 tip 111 to cover the intended area of bone preparation while passing through a small ‘keyhole’ incision in the skin.

The surgical tool guide 100 is configured such that the movement of the working tip 111 of the surgical tool 110 is constrained to a three dimensional volume of space. As such, surgical tool guide 100 can be configured such that the working tip is guided to produce a predefined bone surface geometry.

The three dimensional volume of space can be defined in a pre-surgical planning phase, for example using a computer model of the bone 200. The three dimensional volume of space may be the area which is to be resected on the surface of bone 200.

Once the three dimensional volume of space has been defined, the surgical tool guide 100 can be configured such that the working tip 111 of the surgical tool 110 is constrained in such a way that it may only move within the three dimensional volume of space.

For example, the relative positions of the guide surface, the slot 112, the connecting arms 118, the distal portion 108 of the mechanical guide 102, the pivot means/bearing 116 and the abutment portion 114 of the surgical tool 110 can be specifically designed to constrain the movement of the working tip 111 of the surgical tool 110 to the three dimensional volume of space.

Advantageously, such a configuration means that operations are greatly simplified as a surgeon need only hold and manipulate the proximal end of the surgical tool 110, following the guidance of the slot or slots 112 in conjunction with the pivot means/bearing 116.

It will be understood that the configuration of the surgical tool guide 100 shown in the Figures are merely exemplary. In practice, the configuration and shape of the surgical tool guide 100 can designed according to the type of operation being carried out and/or the three dimensional volume of space which represents the area to be operated on and/or resected by the surgical tool 110.

For example, although the mechanical guide 102 shown in FIG. 1a comprises a slot 112 in the shape of a spiral, other shaped slots could be used, depending on the geometry of the desired three dimensional volume of space to which the working tip 111 of the surgical tool 110 is to be constrained.

Although the upper surface of the proximal portion 106 of the mechanical guide 102 shown in FIG. 1a is domed, the upper surface may comprise any shape suitable for constraining the working tip of the surgical tool 110 to a particular geometry. For example, a much more complex geometry may be used in certain configurations. Alternatively, a flat surface may also be used in certain embodiments.

Although the mechanical guide 102 shown in FIGS. 1a and 1b comprises a single continuous slot 112, multiple slots may be used such that the surgical tool 110 can be removed from a particular slot in the mechanical guide 102 and reinserted into a different slot, each slot constraining the working tip of the surgical tool 110 to a particular portion of the predetermined volume, perhaps with some overlap. The use of multiple slots allows a single surgical tool guide 100 to be used to produce multiple and/or more complex three dimensional volumes of space.

Although the mechanical guide 102 shown in FIG. 1a comprises a grooved slot 112 as a means for guiding the surgical tool 110 by way of abutment of the collar 114 of the surgical tool therewith, other means for guiding the surgical tool 110 are envisaged.

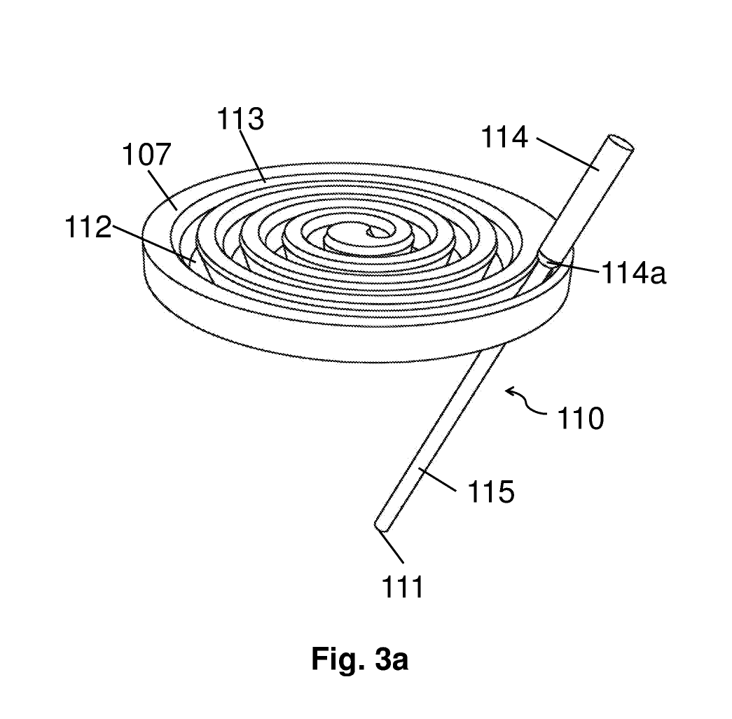

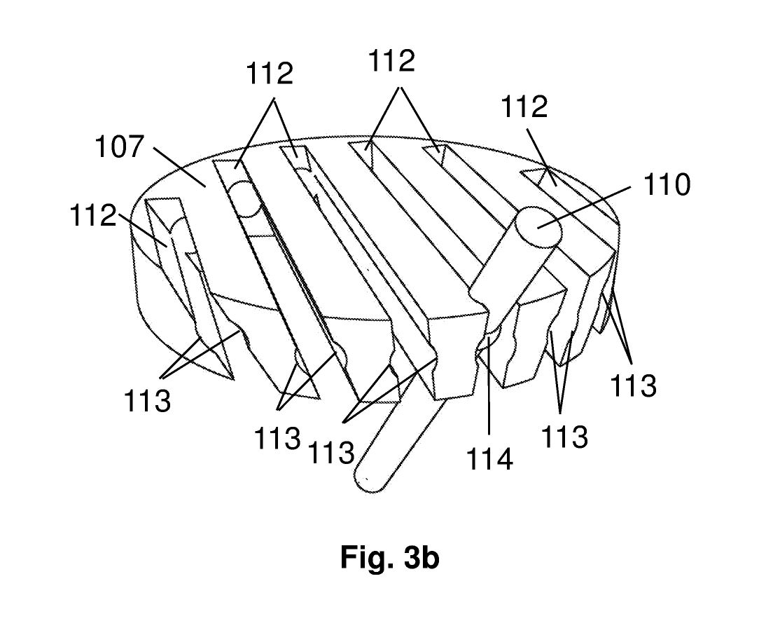

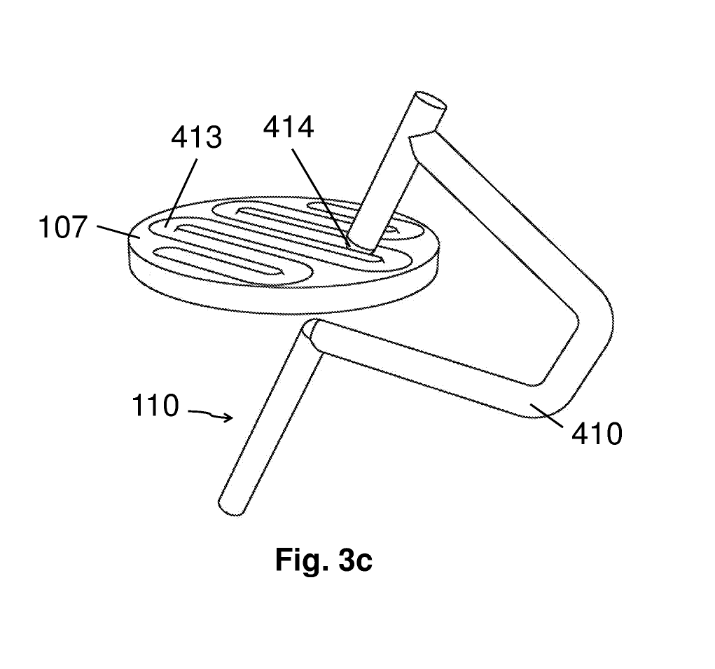

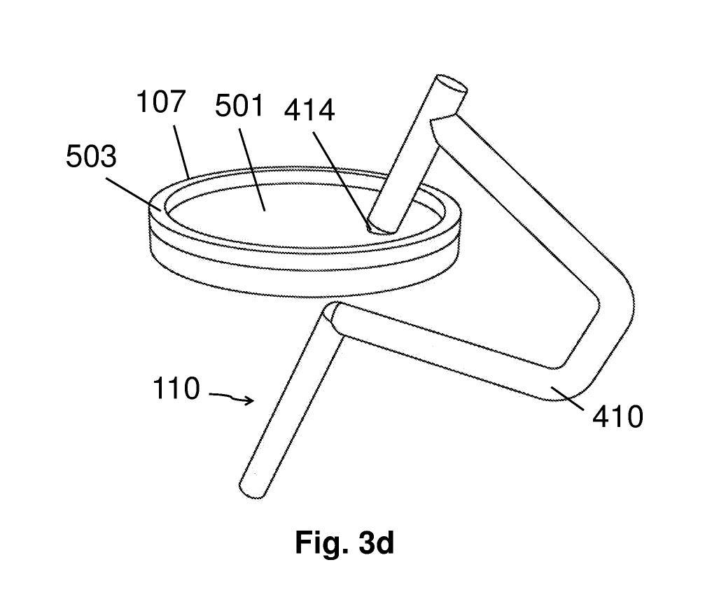

FIG. 3a depicts a guide surface that comprises a continuous spiral slot 112 within a flat, disc-like member 107 and surgical tool 110 disposed therein. The slot 112 comprises a groove 113 with a cross-section which matches a lower end 114a of an abutment portion 114 disposed on the surgical tool 110 such that the lower end 114a of abutment portion 114 can sit within the groove. The groove 113 show in FIG. 3a extends across approximately half the depth of the slot 112.

FIG. 3b depicts an alternative embodiment, in which the guide surface comprises a series of six discrete slots 112 within a planar disc-like member 107, and surgical tool 110 disposed within one of those slots 112. The slots 112 each comprise a groove 113 with a cross-section which matches that of abutment portion 114 disposed on the surgical tool 110 such that the abutment portion 114 can be inserted into the groove 113 of each slot. The grooves 113 shown in FIG. 3b are located approximately halfway down their respective slots 112 such that, when the abutment portion 114 of the surgical tool is disposed within one of the slots 112, movement of the surgical tool 110 is constrained by the slot 112 and its groove 113.

FIG. 3c depicts an alternative guide surface that comprises a continuous serpentine groove 413 on an upper surface of a disc-like member 107 and surgical tool 110 received in the groove. The cross-section of the groove 413 matches that of a corresponding abutment portion 414 of the surgical tool 110. The surgical tool 110 further comprises a profiled portion 410 disposed between the abutment portion 414 and a working tip 111 of the surgical tool (not shown). Abutment of the abutment portion 414 against the groove 413 of the guide surface 107 constrains movement of the surgical tool 110 to the path of the groove 413.

FIG. 3d depicts an alternative guide surface that comprises a disc-like member 107. The disc-like member 107 comprises a smooth upper surface 501 without a slot which is surrounded by a wall 503. A surgical tool 110 is shown with an abutment portion 414 of the surgical tool 110 abutting the smooth upper surface 501. The surgical tool 110 further comprises a profiled portion 410 disposed between the abutment portion 414 and a working tip 111 of the surgical tool (not shown).

A surgeon is able to move the surgical tool 110 across the smooth upper surface 501 freely whilst movement of the surgical tool 110 is still controlled over a defined area and/or depth by the upper surface 501 and wall 503 in conjunction with the distal pivot mean, e.g. the bearing 116.

As an alternative to the profiled portion 410 of the surgical tool 110 shown in FIGS. 3c and 3d, the abutment portion 114 of the surgical tool 110 shown in FIGS. 1a to 3a may be adapted to further comprise a side arm that rested onto the guiding surfaces shown in FIGS. 3c and 3d.

It will be understood that the embodiments of guide surfaces described and depicted herein are only examples of many possible embodiments of guide surfaces of the present invention and are by no means an exhaustive list of possible guide surface embodiments.

It will be understood that various depicted and described configurations of slots, grooves and/or shapes of the various guide surfaces described herein are only examples of many possible configurations. The configurations of the slots, grooves and/or shapes of the various guide surfaces can be chosen and/or designed such that when used as part of a complete surgical tool guide 100, movement of a working tip 111 of a surgical tool 110 used with the surgical tool guide 100 is constrained to a particular area or three dimensional volume of space.

The proximal portion 106 may be configured to be removably attachable to the connecting arms 118, allowing the guide surface to be replaced with a different guide surface.

The surgical tool guide 100 may also be used in conjunction with an appropriate surgical tool 110 for delivery of a material or movement of an imaging or other therapeutic device.

It will be understood that the each of the embodiments of the surgical tool guide described herein may be used in both open surgery and minimally invasive surgery applications. In open surgery applications, the entire area of the bone surface which is to be operated on and upon which the contoured pads 120, 120b sit may be exposed. In minimally invasive surgery applications, each of the contact arms 104, 104b may be configured to pass through a respective arthroscopic incision, as described in detail above.

1. A surgical tool guide for minimally invasive preparation of a bony surface to receive an implant, the surgical tool guide comprising:
at least one contact surface for securely registering the surgical tool guide on a bone; and
a mechanical guide means configured to restrict movement of a surgical tool received therein such that a working tip of the surgical tool, in use, is guided over a predefined area of the bone.

2. The surgical tool guide of claim 1, wherein the mechanical guide means is further configured to restrict movement of the surgical tool to a predetermined depth when in use.

3. The surgical tool guide of claim 1, wherein the contact surface is configured to register the surgical tool guide in a unique position on the bone surface.

4. The surgical tool guide of claim 1, wherein there are a plurality of contact surfaces.

5. (canceled)

6. The surgical tool guide of claim 1, wherein the mechanical guide means is configured, in use, to constrain movement of the working tip of the surgical tool to a three dimensional volume of space defined in a pre-surgical planning phase.

7. The surgical tool guide of claim 1, wherein the mechanical guide means comprises a surface contoured to guide the working tip to produce a predefined bone surface geometry.

8. The surgical tool guide of claim 1, wherein one or more of the contact surfaces is removably attachable to the mechanical guide means, and wherein each of the contact surfaces is removably attachable to the mechanical guide means in such a way that the surgical tool guide can only be assembled in a single configuration.

9. (canceled)

10. The surgical tool guide of claim 1, wherein each of the contact surfaces comprises a contoured pad disposed at a distal end of a contact arm relative to the mechanical guide means, and wherein one or more of the contoured pads is configured to contact a unique position on a bone surface.

11. (canceled)

12. The surgical tool guide of any of claim 1, wherein one or more of the contact surfaces comprises a fixing means selected from a group comprising: wires, pins and screws, securable to the bone, and wherein said fixing means includes depth stops or spacers to locate the mechanical guide means at a known height above the bone.

13. (canceled)

14. The surgical tool guide of claim 1, wherein the mechanical guide means comprises a proximal portion and a distal portion, wherein the proximal portion comprises a guide surface to restrict the depth and position of the surgical tool when in use and, preferably, and wherein the distal portion comprises a pivot means to restrict movement of the surgical tool when in use.

15. The surgical tool guide of claim 14, wherein the guide surface comprises one or more slots through which a surgical tool can be inserted.

16. The surgical tool guide of claim 14, wherein the guide surface comprises a groove into which a part of the cutting device can be inserted when in use.

17. The surgical tool guide of claim 14, wherein the distal portion comprises an aperture through which a surgical tool is inserted when in use.

18. The surgical tool guide of claim 17, wherein the distal portion comprises a bearing containing said aperture.

19. The surgical tool guide of claim 1, wherein each of the contact surfaces are designed to be passed through a respective incision.

20-21. (canceled)

22. The surgical tool guide of claim 1, wherein the mechanical guide means comprises a removable guide plate which can be interchanged with at least one other guide plate.

23. A surgical kit comprising:
a surgical tool guide comprising:
at least one contact surface for securely registering the surgical tool de on a bone;
and
a mechanical guide means configured to restrict movement of a surgical tool received therein such that a working tip of the surgical tool, in use, is guided over a predefined area of the bone;
and
a surgical tool configured to engage with the mechanical guide means of the surgical tool guide.

24. The kit of claim 23, wherein the mechanical guide means comprises a proximal portion and a distal portion, wherein the proximal portion comprises a guide surface to restrict the depth and position of the surgical tool when in use and, preferably, wherein the distal portion comprises a pivot means to restrict movement of the surgical tool when in use, wherein the guide surface comprises a groove into which a part of the cutting device can be inserted when in use, and wherein the surgical tool comprises a protrusion for abutment against the groove of the mechanical guide means.

25. The kit of claim 23, wherein the mechanical guide means comprises a proximal portion and a distal portion, wherein the proximal portion comprises a guide surface to restrict the depth and position of the surgical tool when in use and, preferably, wherein the distal portion comprises a pivot means to restrict movement of the surgical tool when in use, wherein the guide surface comprises one or more slots through which a surgical tool can be inserted wherein the mechanical guide means comprises a collar received in the slot, and wherein the collar is configured to limit the depth of the surgical tool therein.

26-27. (canceled)

28. A method of manufacturing a surgical tool guide the surgical tool guide comprising at least one contact surface for securely registering the surgical tool guide on a bone, and a mechanical guide means configured to restrict movement of a surgical tool received therein such that a working tip of the surgical tool, in use, is guided over a predefined area of the bone, the method comprising the steps of:
designing the at least one contact surface on the basis of patient-specific data such that the surgical tool guide is securely registrable in a predefined position and orientation on the patient's bone;
designing the mechanical guide means on the basis of patient-specific data such that a working tip of a surgical tool, in use, is guided over a predefined area of the patient's bone defined in a pre-surgical planning phase; and
manufacturing the surgical tool guide according to said designing steps.
